(19) United States
(12) Patent Application Publication
(10) Pub. No.: US 2017/0000373 A1
(43) Pub. Date: Jan. 5, 2017

(54) METHODS, SYSTEMS, AND DEVICES FOR ASSESSING NEUROMUSCULAR STATUS OF LIGAMENTOUS AND SOFT TISSUES

(71) Applicant: Wake Forest University Health Sciences, Winston-Salem, NC (US)

(72) Inventors: Ian D. Hutchinson, Winston-Salem, NC (US); Thorsten M. Seyler, Winston-Salem, NC (US)

(21) Appl. No.: 14/790,791

(22) Filed: Jul. 2, 2015

(51) Int. Cl. A61B 5/0488; A61B 5/00

(52) U.S. Cl. CPC A61B 5/0488; A61B 5/4041; A61B 5/6867

(57) ABSTRACT

Systems, devices, and methods for neuromuscular testing of ligamentous or soft tissue structures in mammals provide information (e.g., EMG feedback) about the status of neuromuscular function and reflex activity for, e.g., joint structures, in consideration of surgical or other procedures.

### FIELD

The present invention relates to assessing the neuromuscular status of mammalian joint structures.

### RELATED APPLICATIONS

The present application claims priority to U.S. Provisional Patent Application No. 61/020,493, filed Jul. 3, 2014. The disclosure of U.S. Provisional Patent Application No. 61/020,493 is hereby incorporated by reference in its entirety herein.

### BACKGROUND

The efficacy of a surgical procedure can be dramatically influenced by the status of the underlying tissue. Unfortunately, there are often not methods available for assessing the ability of a patient to respond to certain surgical interventions. For example, it is known that reflex phenomena mediate normal joint loading by mediating active muscle forces. Still, neuromuscular testing of ligamentous and soft tissue structures that contribute to joint stability and proprioception is not presently measured before or during joint replacement or soft tissue reconstruction. Hence, soft tissue reconstruction and joint replacements are currently undertaken without consideration of the peri-articular neuromuscular status of the joint.

As an example, Anterior Cruciate Ligament (ACL) reconstruction is a common orthopedic intervention with over 100,000 procedures performed annually in the United States. Neuromuscular status is inadequately considered for these procedures to date. Thus the orthopedics field stands to benefit from the ability to describe and develop objective measures of neuromuscular joint status, so as to assess reflex reactivity of stabilizing muscle groups to a stimulus. Objective measures can allow assessment of the ability of the ligaments and soft tissues to contribute to joint loading and stability by mediating active muscle thereby facilitating individualization of surgical treatment regimens and possibly also individualization of rehabilitation programs.

### SUMMARY

Embodiments of the present invention include methods, devices and systems for assessing the neuromuscular status of mammalian joint structures. The invention may be embodied in a variety of ways.

In some embodiments, the present invention comprises a method for testing neuromuscular status of a ligamentous or soft tissue structure in a mammal, comprising: stimulating a ligamentous or soft tissue structure; observing EMG output to determine a quantitative measure of neuromuscular status for the ligamentous or soft tissue structure; and using the quantitative measure of neuromuscular status to guide medical treatment related to the ligamentous or soft tissue structure. In an embodiment, the medical treatment is a surgical intervention.

In some embodiments, the invention comprises a device for testing neuromuscular status of a mammalian joint including a component for stimulating a ligamentous or soft tissue structure, a component for detecting neuromuscular reflex activity to generate electromyographic (EMG) output, and a processing unit component to receive and process the EMG data. In some embodiments, the device further includes a display for a quantitative measure regarding the neuromuscular status of the ligamentous or soft tissue structure

In some embodiments, the invention comprises a system for testing neuromuscular status of a ligamentous or soft tissue structure in a mammal, including a stimulating component, a processing component, and a feedback component to indicate neuromuscular status of the ligamentous or soft tissue structure. In some embodiments, the stimulating component comprises a device for electrical stimulation.

### BRIEF DESCRIPTION OF FIGURES

The present invention may be better understood by referring to the following non-limiting figures.

FIG. 1 shows an example of a flow chart outlining steps for assessing neuromuscular status of a ligament or other soft tissue, according to one embodiment of the invention.

FIG. 2 shows an example of a flow chart outlining steps for assessing neuromuscular status of the anterior cruciate ligament (ACL) in the knee joint, according to one embodiment of the invention.

FIG. 3 shows an anterior view of the knee joint, with a view of the anterior cruciate ligament (ACL) in contact with a stimulator, according to one embodiment of the invention.

FIG. 4 shows a device for neuromuscular testing, according to one embodiment of the invention.

FIG. 5 shows configurations for transmitting and processing data generated in neuromuscular testing (e.g., EMG data).

### DETAILED DESCRIPTION

The methods, systems, and devices of the present invention may be advantageously utilized in treatment protocols for humans, and/or in veterinary applications. Objective neuromuscular testing of innervated anatomical structures may facilitate individualization of surgical treatment regimes, based on functional status of the structures (e.g., comprised in a synovial joint) in the individual patient.

The following description recites various aspects and embodiments of the present invention. No particular embodiment is intended to define the scope of the invention. Rather, the embodiments merely provide non-limiting examples of various methods and systems that are at least included within the scope of the invention. The description is to be read from the perspective of one of ordinary skill in the art; therefore, information well-known to the skilled artisan is not necessarily included.

### DEFINITIONS

Where the definition of terms departs from the commonly used meaning of the term, applicant intends to utilize the definitions provided below, unless specifically indicated.

For purposes of the present invention, it should be noted that the singular forms, “a,” “an” and “the,” include reference to the plural unless the context as herein presented clearly indicates otherwise.

Electromyography (EMG), as used herein, refers to a diagnostic procedure to assess the health of muscles and the nerve cells that control them (motor neurons). Motor neurons transmit electrical signals that cause muscles to contract. An EMG translates these signals into graphs, sounds or numerical values that a specialist interprets. An EMG may utilize electrodes to transmit and/or detect electrical signals.

During a needle EMG, a needle electrode inserted directly into a muscle records the electrical activity in that muscle. A nerve conduction study, another aspect of an EMG, uses electrodes taped to the skin (surface electrodes) to measure the speed and strength of signals traveling between two or more points. EMG results can reveal nerve dysfunction, muscle dysfunction or problems with nerve-to-muscle signal transmission.

Neuromuscular, as used herein, refers to the interaction of sensory elements from muscles and other soft tissues and motor elements of muscle function, affecting locomotion and joint function.

Positive reflex and negative reflex, as used herein, refer to presence and absence, respectively, of positive muscle activation(s) as the result of peripheral stimulation (electrical or mechanical) of other soft tissues or at another anatomical site. Presence of a positive reflex may be determined based on EMG feedback in response to stimulus. Validation of a positive reflex may be determined based on observation of muscle contraction.

### Methods for Using Quantitative Neuromuscular Status to Guide Medical Treatment

In one aspect, the invention comprises methods for objective neuromuscular testing for reflex arcs through direct stimulation of the ligaments or soft tissue structures and using quantitative measures of neuromuscular status to guide medical treatment decisions related to the ligamentous or soft tissue structure. Such testing may be particularly advantageous prior to surgery, e.g., knee or shoulder surgery. In some embodiments, the testing comprises electrical stimulation of the structure and/or physical manipulation of the joint comprising the structure. In some embodiments, the invention comprises measuring electromyographic (EMG) feedback as a function of reflex activity.

Neuromuscular reflex testing of ligaments as a routine diagnostic aid during joint arthroplasty and soft tissue reconstruction has not been described. The present invention recognizes that neuromuscular status may be correlated with surgical outcomes and thereby used as a factor to guide or direct treatment. In some embodiments, the present invention comprises a numerical indication of reflex latency. Alternatively (or additionally), in some embodiments the presence or absence of reflex activity may provide a binary outcome measure for optimal efficiency in a surgical setting.

In some aspects, embodiments of the invention may allow surgeons or other healthcare providers to capture quantifiable objective evidence of neuromuscular status in operated joints to guide design of, e.g., a prosthesis, and to predict outcome in arthroplasty and soft tissue reconstruction. Thus the neuromuscular status of a ligament or other soft tissue structure reflects the function of the nerves in conducting a signal into the associated muscle(s).

Correlating surgical outcomes with neuromuscular status assessments of joint structures prior to intervention through surgical procedures may facilitate the physician's ability to personalize the treatment plan before, during and after the surgical procedure. Assessment of neuromuscular status in conjunction with surgical procedures may be developed for all the synovial joints, and particularly knee, shoulder, and ankle.

In some embodiments, the ligament or soft tissue to be tested is identified using the arthroscope or is carefully exposed using an open arthrotomy. In some embodiments, testing may be performed in advance of the day of surgery, for example in an office setting facilitated by concomitant use of minimally invasive arthroscopic equipment. Regardless of the method used to gain access to the ligament, a stimulator is then attached to the ligament or soft tissue, and an electrical stimulus is applied.

In some embodiments, the structure being tested is a ligamentous or soft tissue structure within a joint. In some embodiments, the joint is a mammalian joint. In some embodiments, the joint is a human joint. In some embodiments, the joint is canine, feline, or equine. In some embodiments, the joint may be a synovial joint (e.g., knee, shoulder, or ankle). In some embodiments, the joint may be identified as a candidate for a surgical procedure. In some embodiments, the joint may be designated for an arthroscopic surgical procedure. In some embodiments, the joint may be designated for an open surgical procedure.

Some embodiments of the present invention utilize electrodes to detect neuromuscular status. Electrodes placed in various positions (e.g., skin or needle electrodes) may detect action potentials resulting from electrical stimulation of ligamentous or soft tissue structures. Changes from baseline activity may include changes in amplitude and/or duration of muscle action potentials. Latency may also be affected, as healthy nerves are able to transmit signals faster than damaged or dysfunctional nerves. In some embodiments the feedback generates an indicator of reflex latency.

In some embodiments, quantitative EMG feedback may be transmitted in an absolute readout, indicating latency in numerical terms (e.g., 120 ms). In some embodiments, designated latency thresholds may generate a quantitative measure of neuromuscular status as binary output. Binary output may indicate fully functional neuromuscular status or impaired status. For example, the feedback may generate a readout within a designated spectrum to read in green and anything outside the spectrum to read in red (e.g., displaying real numbers in the appropriate color, or just the binary indicator without real numbers). In some embodiments, a signal readout screen icon may be included to facilitate monitoring by healthcare personnel who are red-green color blind. Or other binary output formats are possible.

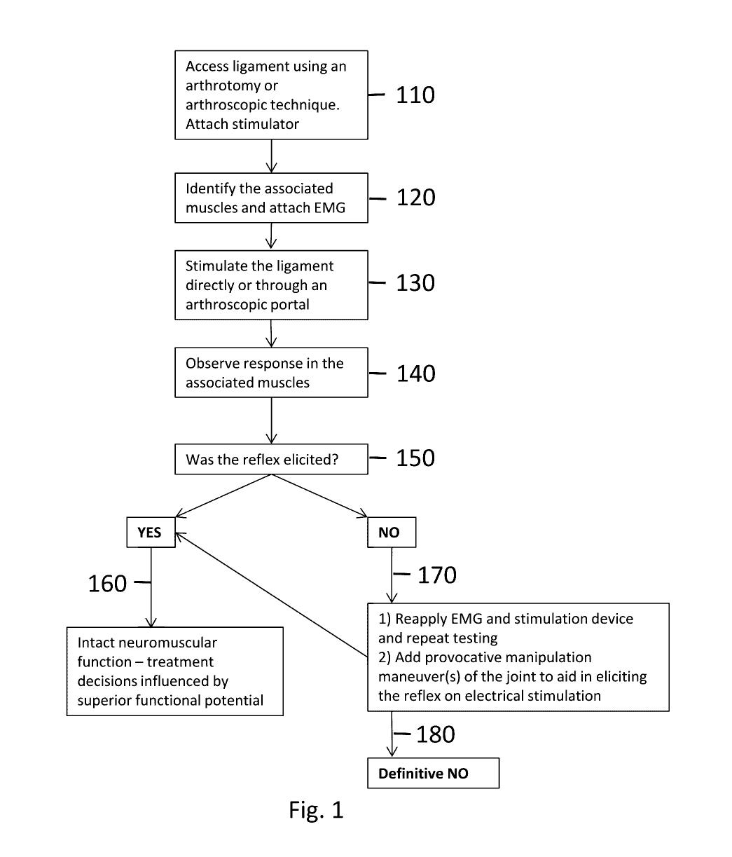

FIG. 1 illustrates and example embodiment of a method of the invention. In the first step 110, a surgeon or other healthcare provider gains access to the ligament or soft tissue of interest, e.g., via arthrotomy or arthroscopy and an electrical stimulator is attached to the ligament or tissue. Next, the muscles attached to the ligament and/or other soft tissue are identified 120 and electrodes for detecting/recording EMG activity are attached (e.g., to the skin covering the muscles). An electrical stimulus is applied to the ligament or tissue of interest via the stimulator 130 and healthcare personnel observe the muscles and EMG data 140 for positive reflex indicators. EMG activity and/or contraction of muscles indicates that the reflex was elicited 150 and therefore the neuromuscular function of the ligament or soft tissue is intact 160 and may have superior functional potential. The positive reflex may lead the surgeon to choose a procedure and prosthesis that preserve and utilize the functional ligament. In the absence of either indicator 170, the stimulator and/or EMG electrodes are adjusted or reapplied and the stimulation is repeated. If there is still no response, the joint is manipulated for mechanical stimulation (e.g., manually apply flexing and/or extension to a joint). If the healthcare providers continue to observe no response, the conclusion is negative reflex and compromised or absent neuromuscular function 180. As a result of this definitive NO, for example, a surgeon who is considering repairing the ligament and/or soft tissue, may instead choose a replacement procedure with an artificial joint.

The methods of the invention may be used for any ligament and/or other soft tissue. Further non-limiting descriptions of the use of such methods for knee and/or shoulder joints is provided below.

### ACL and PCL

The intact ACL contains mechanoreceptors capable of detecting changes in tension that facilitate somatosensory feedback regarding the speed and direction, acceleration and position of the knee joint motion during dynamic activities.

Reflex activity of the ACL has been demonstrated in humans following direct electrode and physical stimulation of the ACL. During the late stance phase of gait, the hamstrings co-contract against the quadriceps to decelerate knee extension and provide active stabilization of the knee. This occurs at low knee flexion angles when the ACL is most stretched and is normally facilitated by a neuromuscular phenomenon known as the “ACL-hamstring reflex arc,” described as a protective ligament-muscular pathway.

The protective reflex may be absent or deficient where neuromuscular status is compromised, and presence or absence of the protective reflex affects the surgical patient's ability to manage various prosthetic designs as well as their recovery and rehabilitation following surgery.

Neuromuscular status has been evaluated to investigate re-innervation following ACL reconstruction. However, the relationship between ACL neuromuscular status and knee joint function as it relates to knee joint replacement or soft tissue/cartilage reconstruction has not been described.

Currently, the cruciate ligaments of the knee are practically considered as passive stabilizers of knee joint stability by the orthopedic surgeon, and assessment in the operating room consists of visual inspection and rudimentary tensile examination. Knowing the functional status of nerves in a ligament can help the surgeon better understand the potential for preserving that ligament, for example. Thus, assessment of neuromuscular status of ligaments or other soft tissue structures may facilitate better design of treatment plans (e.g., design of a prosthesis and/or rehabilitation program).

Neuromuscular assessment of the anterior cruciate ligament (ACL) or posterior cruciate ligament (PCL) in arthroplasty or soft tissue joint repair may provide a physician with information about the status of the joint structures that allows consideration of functional status for the individual patient with regard to the extent of the arthroplasty and the appropriate level of constraint for a prosthesis.

In addition, for soft tissue reconstructions in the knee (e.g., articular cartilage and meniscus repair/replacement), neuromuscular assessment may assist in identifying patients that have higher risk of failure and may further assist in designing rehabilitation protocols.

In some embodiments of the present invention, neuromuscular testing of joint structures (e.g., ACL or PCL) may present actionable information as correlations with surgical outcomes are investigated and more thoroughly understood. As such, routine testing of neuromuscular status in joint structures as proposed herein presents the opportunity to better refine a treatment plan for the individual patient. Moreover, customized devices and protocols for such testing may facilitate use of routine neuromuscular testing in the surgical setting.

For example, the goal of ACL reconstruction is to restore the native function of ACL by situating a replacement graft between the femoral and tibial insertion sites with appropriate graft orientation and tensioning. Ultimately, the aim is that the graft will successfully undergo “ligamentization”—a process describing cellular infiltration, collagen microarchitecture reorganization and vascular invasion in the graft and re-creation of the native ACL biomechanical properties.

Direct stimulation of the ACL with monitoring of the hamstring muscles for the presence of a protective reflex may enable surgeons to more accurately choose the extent and design of arthroplasty modality and may assist with designing/directing rehabilitation following soft tissue reconstruction. Thus, in some embodiments, visible contraction of the hamstring may be observed simultaneously with EMG feedback.

In another example, the PCL contributes to active muscle joint stabilization and lower limb tone. The role of the PCL in stabilization has not been described fully. However, reflex activity has been elicited following direct electrical stimulation of the PCL. Neuromuscular function of the PCL may contribute to mid flexion stability in PCL retaining arthroplasty. Thus, various knee procedures, such as ACL and PCL procedures, may incorporate neuromuscular testing as a routine objective measure of joint status.

In another type of procedure, ligament-sparing knee joint arthroplasty/unicompartmental/robotic-assisted arthroplasty, neuromuscular testing of the anterior cruciate ligament (ACL) and posterior cruciate ligament (PCL) may help to identify patients that may benefit from:

i) less constrained/minimally invasive interventions (e.g., prosthesis design) in the presence of intact ACL reflex function, and

ii) PCL retaining designs where the PCL neuromuscular innervation is intact for patient comfort in mid-flexion.

In additional examples, neuromuscular testing according to some embodiments of the invention may also be indicated for PCL-sparing arthroplasty and for the coracoacromial ligament in the shoulder as a prognostic indicator of shoulder neuromuscular status in shoulder surgery, including arthroplasty.

In arthroplasty procedures, neuromuscular assessment may allow for design of a more suitable prosthesis in terms of level of activity versus constraint, ultimately resulting in improved patient satisfaction.

In soft tissue reconstruction procedures, neuromuscular assessment according to embodiments of the invention may provide a prognostic indicator and guide neurorehabilitation and expectations for weight-bearing and/or return to sport and/or return to full activity.

The methods, systems, and devices described herein may be used (e.g., on the ACL) in the setting of unicompartmental/robot assisted arthroplasty and soft tissue reconstruction.

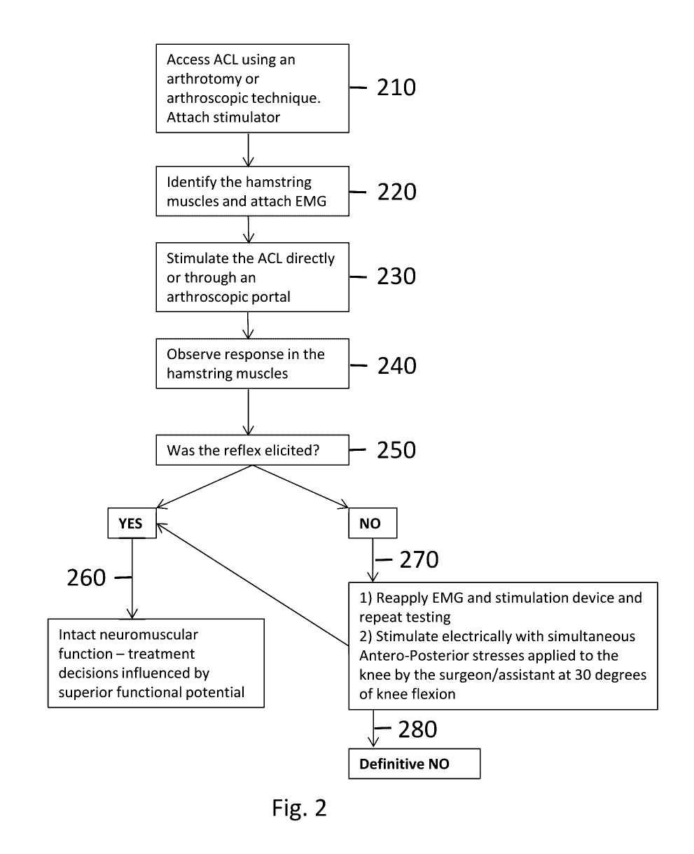

FIG. 2 is a schematic showing an example of steps for performing neuromuscular assessment of the ACL, according to one embodiment of the invention. In the first step 210, a surgeon or other healthcare provider gains access to the ACL via arthrotomy or arthroscopy, and a stimulator is attached to the ACL. Next, the hamstring muscles are identified 220 and electrodes for detecting/recording EMG activity are attached (e.g., to the skin covering the muscles). An electrical stimulus is applied to the ACL via the stimulator 230 and healthcare personnel observe the muscles and EMG data 240 for positive reflex indicators. EMG activity and/or contraction of muscles indicates that the reflex was elicited 250 and therefore the neuromuscular function of the ACL is intact 260 and may have superior functional potential. The positive reflex may lead the surgeon to choose a procedure and prosthesis that preserve and utilize the functional ligament (e.g., bicruciate-retaining). In the absence of either indicator 270, the stimulator and/or EMG electrodes are adjusted or reapplied and the stimulation is repeated. If there is still no response, the joint is manipulated for mechanical stimulation (e.g., manually apply 30 degrees flexion in antero-posterior stress examination). If the healthcare providers continue to observe no response, the conclusion is negative reflex and compromised or absent neuromuscular function 280. As a result of this definitive NO, for example, a surgeon who is considering a bicruciate-retaining arthroplasty may opt instead for conventional total knee arthroplasty.

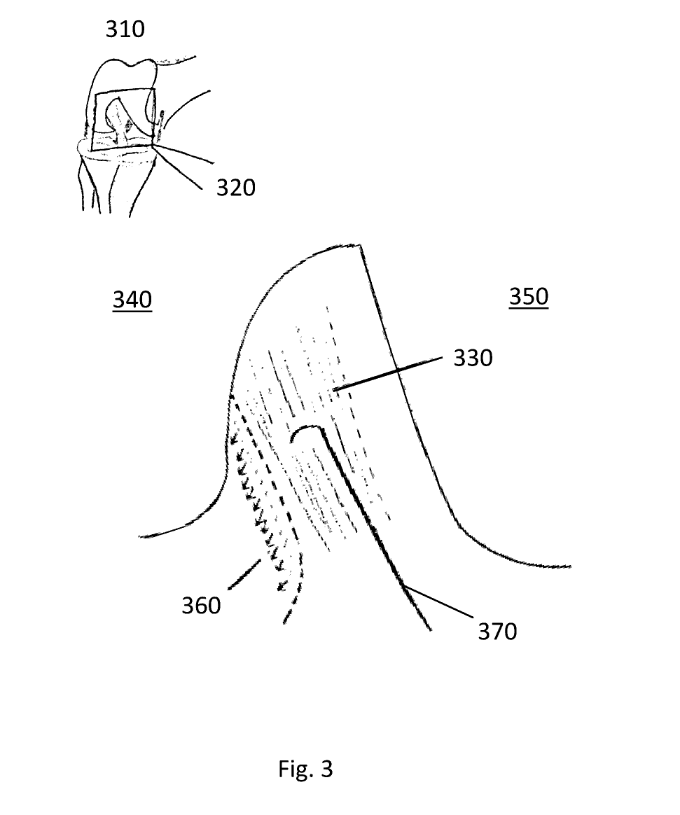

FIG. 3 is an illustration of the anterior view of the right knee 310, focusing on the intercondylar fossa 320 and the ACL 330. The lateral aspect is at left 340 and medial aspect at right 350. The infrapatellar synovial fold (ligamentum mucosum) 360 is shown attaching anteriorly to the synovial membrane/anterior capsule. A stimulator is attached to the ACL via a hook 370 for generating electrical stimulus as described in FIG. 2.

### Coracoacromial Ligament

In the shoulder joint, the coracoacromial ligament reflex results in a general inhibition of voluntarily activated shoulder muscles and is believed to contribute to muscle coordination and functional joint stability in the shoulder. Current clinical outcomes comparing surgery to physical therapy alone are equivocal in patients with rotator cuff tears. It follows that a lack of comprehensive, objective neuromuscular assessment of the shoulder may be responsible for the failure to identify the most suitable candidates for surgery. For shoulder surgery, assessment of the coracoacromial ligament reflex may be developed as a predictor of success in soft tissue reconstruction (e.g., rotator cuff surgery) and total/partial shoulder replacement including reverse shoulder arthroplasty.

For example, cuff repairs are undertaken in patients to provide symptomatic relief, restoring the joint anatomy and improving range of motion. However, it is currently unclear why certain patients benefit from this procedure more than others, and the incidence of re-tears has necessitated intense basic science and clinical research. Neuromuscular assessment of the shoulder prior to performing surgery can identify patients that have an intrinsic advantage to protecting their repair and may assist in identifying candidates that will benefit from surgery. In addition, assessment in the post-operative period can identify patients that are recovering neuromuscular function, for example after a rotator cuff repair. Such assessment can also identify areas of the shoulder tissue that are not responding to repair, and allow for development of physical therapy regimens and/or other treatment protocols to facilitate recovery. Similarly, a SLAP tear (Superior Labrum from Anterior to Posterior, which occurs at the point where the tendon of the biceps muscle inserts on the labrum) necessitates soft tissue repair of the cuff, presenting a specific instance where neuromuscular assessment may be beneficial in patient selection and post-operative rehabilitation.

### Devices

In some embodiments for neuromuscular reflex testing of ligaments or soft tissues, the invention comprises a stimulating device. In some embodiments, the stimulating device is a hand-held device. In some embodiments, such a hand-held device may be held in the healthcare provider's hand and may be similar (e.g., in size and/or dimension) to a probe, needle, or writing instrument.

In some embodiments, the structure being tested is a ligamentous or soft tissue structure within a joint. In some embodiments, the joint is a mammalian joint. In some embodiments, the joint is a human joint. In some embodiments, the joint is canine, feline, or equine. In some embodiments, the joint may be a synovial joint (e.g., knee, shoulder, or ankle). In some embodiments, the joint may be identified as a candidate for a surgical procedure. In some embodiments, the joint may be designated for an arthroscopic surgical procedure. In some embodiments, the joint may be designated for an open surgical procedure.

In some embodiments, the stimulation generates feedback. In some embodiments, the feedback is electromyographic. In some embodiments, the feedback is contraction of a muscle or muscle group (i.e., observing positive EMG feedback with or without visible muscle contraction in response to the stimulus is a positive reflex). In some embodiments, the EMG feedback is automatically analyzed by the system to generate useful output signals. In some embodiments of a system for testing neuromuscular status, the feedback is detected and processed to generate a numerical read for viewing by healthcare personnel, in a visual output. In some embodiments, the visual output is binary. In some embodiments, a binary output is indicated via audio or other non-visual signals or indicators.

In some embodiments, following stimulation of ligamentous or soft tissue structures (e.g., electrically or by physical manipulation), feedback may be generated and observed in several different ways. For example, surface EMG may be detected via skin electrodes, intramuscular EMG may be detected via several different types of needle electrodes inserted into muscle tissue, and/or visual observation of muscle reflex may indicate intact neuromuscular function.

In some embodiments, EMG feedback may be transmitted in an absolute readout, indicating latency in numerical terms (e.g., 120 ms). In some embodiments, designated latency thresholds may generate a binary output. For example, the feedback may generate a readout within a designated spectrum to read in green (e.g., giving real numbers) and anything outside the spectrum to read in red. In some embodiments, a signal readout screen icon may be included to facilitate monitoring by healthcare personnel who are red-green color blind.

In some embodiments, a hand-held device comprises a stimulating member (e.g., Teflon-coated platinum iridium wire, Teflon-insulated stainless steel wires with 3-mm exposed tips) which may be attached or anchored to a ligamentous structure or soft tissue to be stimulated. A hook, clamp, or other member may be customized for a particular application (e.g., a clamp designed for attaching to and stimulating the ACL).

For example, a handheld nerve stimulator (e.g., Checkpoint Surgical, Model #9014 Surgical Nerve Stimulator/Locator or B. Brain Medical, Stimuplex HNS12 Peripheral Nerve Stimulator with SENSe) may be used in the intraoperative assessment of the reflex arc by direct stimulation of the ligament. Stimulation of a normally functioning ligament with a stimulus intensity of, e.g., 0.5 mA and 50-100 microseconds may be performed initially (this is expected to generate contraction of the hamstring muscles, e.g., semitendinosus muscle). EMG feedback may be registered via skin or needle electrodes or some combination thereof.

In some embodiments, a device comprising a stimulating component is customized or customizable for arthroscopic applications. For example, a stimulating device may further comprise an adapting extension for electrical stimulation in arthroscopic procedures. In some embodiments, such an adapting extension is insulated. In some embodiments, a device may comprise additional components, such as an element to provide for electrical stimulation during saline insufflation of the joints during arthroscopy.

In some embodiments, a hand-held stimulator device may be reusable. In some embodiments, attachments for ligament or soft tissue stimulation and/or for receiving feedback for EMG measurements (e.g. electrodes) designed for arthroscopic and open procedures may be disposable. In some embodiments, various attachments may be interchangeable (i.e., modular).

In some embodiments, an attachment for stimulation may be designed to contact a ligamentous or soft tissue structure without damaging the structure. In some embodiments, a device may comprise a stimulating member that may hook or loop around nerve fibers to anchor the stimulator to a structure (e.g., ACL) without damaging it. In some embodiments, a stimulating attachment may contact the ligament so as to anchor into the vertically oriented fibers.

A component for detecting electrical activity in muscles (e.g., EMG) may also be included in a device for neuromuscular testing. In some embodiments surface electrodes may be applied to the skin over the muscles being tested. In other embodiments needle electrodes may be inserted into muscles for intramuscular recordings. In some embodiments a combination of surface and needle electrodes may be employed.

In some embodiments, a device may also include a processing unit. The processing unit may be used in conjunction with the hand-held stimulator component of the device to electrically stimulate the ligament arthroscopically or during an open procedure, and EMG feedback may be mediated through direct needle electrode measurement in the respective muscle groups via external leads connected to the processing unit within the sterile field.

Thus in some embodiments a device for neuromuscular testing may include a processor and other components for receiving and processing the EMG data and generating output information in a variety of formats (e.g., within a processing unit).

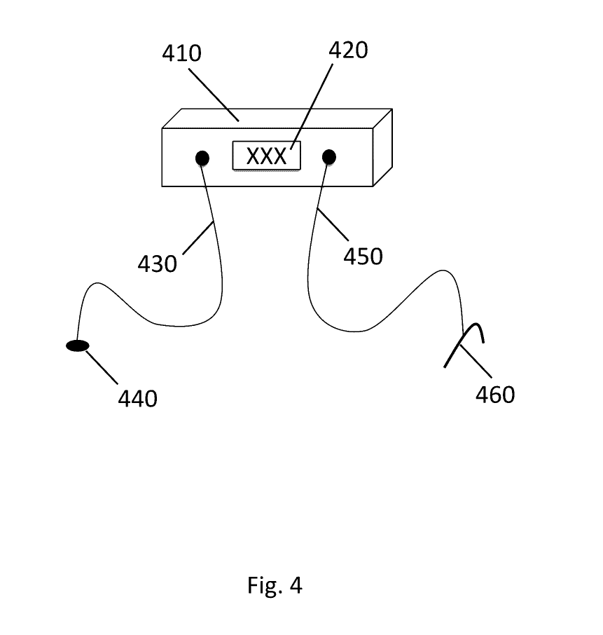

FIG. 4 illustrates a device for neuromuscular testing according to one embodiment of the invention. A processing unit 410 with a display 420 is connected by an input lead 430 to one or more electrodes 440 that receive EMG data from muscles associated with the ligament or other soft tissue structure being tested. An output lead 450 is also connected to the processing unit 410 and the stimulating component of the device 460, for stimulating a ligament or other soft tissue structure. Here, as in FIG. 3, a hook is shown as the stimulating member.

In some embodiments, EMG feedback may be transmitted in an absolute readout, indicating latency in numerical terms (e.g., 120 ms). In some embodiments, designated latency thresholds may generate a binary output. For example, the feedback may generate a readout within a designated spectrum to read in green (e.g., giving real numbers) and anything outside the spectrum to read in red. In some embodiments, a signal readout screen icon may be included to facilitate monitoring by healthcare personnel who are red-green color blind.

Thus in some embodiments, a device for neuromuscular testing may comprise multiple components. A device may include a stimulator (e.g., electrode with hook), a component for generating an electrical impulse to stimulate tissue in contact with the stimulator, a component for receiving an electrical signal in electronic communication with the stimulator; and a user interface including components for entering input and observing output (visually or otherwise).

Thus, in some embodiments, devices for arthroscopic and/or open protocols may be customized to objectively examine neuromuscular function of ligamentous and/or soft tissue structures in, e.g., the knee, shoulder, or ankle during soft tissue repair and arthroplasty procedures, as they relate to active muscle stabilization of the joint following the repair or ligament-sparing joint replacement.

### Systems

In some aspects, the invention is embodied in a system for testing neuromuscular status of a ligamentous or soft tissue structure, comprising a stimulating component and a feedback-detecting component. Such systems may include any or all of the devices described herein. In some embodiments the stimulating component generates electrical stimulation. In some embodiments, the stimulating component is generated through physical manipulation of a joint, wherein the joint comprises the ligamentous or soft tissue structure to be tested. In some embodiments, the stimulating component may be comprised in a hand-held device.

Thus, in some embodiments, a system comprising a hand-held stimulating device incorporates both the ability to deliver an electrical stimulus to a ligament or soft tissue structure and the ability to detect feedback (e.g., EMG) from a select muscle or muscle group(s) to assess the presence of reflex activity and/or reflex latency.

In some embodiments of a system for testing neuromuscular status of a ligamentous or soft tissue structure, the feedback-detecting component comprises electrodes. In some embodiments, electrodes are positioned so as to detect reflex activity in a muscle or muscle group to which the joint is attached. In some embodiments, the electrodes are skin electrodes. In other embodiments, the electrodes are needle electrodes. Or some combination of electrodes may be used.

In some embodiments, the structure being tested is a ligamentous or soft tissue structure within a joint. In some embodiments, the joint is a mammalian joint. In some embodiments, the joint is a human joint. In some embodiments, the joint is canine, feline, or equine. In some embodiments, the joint may be a synovial joint (e.g., knee, shoulder, or ankle). In some embodiments, the joint may be identified as a candidate for a surgical procedure. In some embodiments, the joint may be designated for an arthroscopic surgical procedure. In some embodiments, the joint may be designated for an open surgical procedure.

In some embodiments, the stimulation generates feedback. In some embodiments, the feedback is electromyographic. In some embodiments, the feedback is contraction of a muscle or muscle group (i.e., observing positive EMG feedback with or without visible muscle contraction in response to the stimulus is a positive reflex). In some embodiments, the EMG feedback is automatically analyzed by the system to generate useful output signals. In some embodiments of a system for testing neuromuscular status, the feedback is detected and processed to generate a numerical read for viewing by healthcare personnel, in a visual output. In some embodiments, the visual output is binary. In some embodiments, a binary output is indicated via audio or other non-visual signals or indicators.

In some embodiments, following stimulation of ligamentous or soft tissue structures (e.g., electrically or by physical manipulation), feedback may be generated and observed in several different ways. For example, surface EMG may be detected via skin electrodes, intramuscular EMG may be detected via several different types of needle electrodes inserted into muscle tissue, and/or visual observation of muscle reflex may indicate intact neuromuscular function.

In some embodiments, EMG feedback may be transmitted in an absolute readout, indicating latency in numerical terms (e.g., 120 ms). In some embodiments, designated latency thresholds may generate a binary output. For example, the feedback may generate a readout within a designated spectrum to read in green (e.g., giving real numbers) and anything outside the spectrum to read in red. In some embodiments, a signal readout screen icon may be included to facilitate monitoring by healthcare personnel who are red-green color blind.

In some embodiments, customized software may facilitate use of the systems, devices, and methods described herein. In some embodiments, applications for phones, computer systems and tablets may further facilitate uses of the many embodiments of the invention.

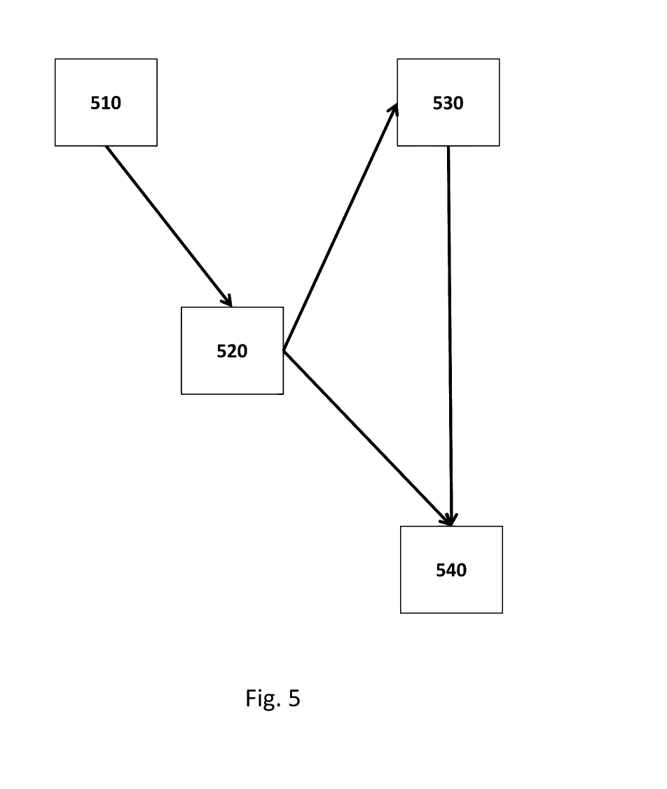

FIG. 5 is a schematic showing possible configurations according to some embodiments of the invention. The stimulating and detecting electrodes 510 may be in communication with a device near the site of the patient 520. This device 520 may register EMG measurements, and it may contain a processor for quantitatively evaluating the measurements as well (i.e., a processing unit). The device 520 may also provide output in visual or other formats, such as a numerical display or binary output (yes-no, or red-green, e.g.). Alternatively, the registering device 520 may not include a processor, and may send the EMG measurements to a remote server or web-based application 530, or directly to any computing device. The EMG data processing could be performed at the remote server or web-based application 530, or by any computing device 540. Results from data processing may be sent by the registering device 520 or the remote server or web-based application 530 directly to any computing device 540.

Methods of the present invention may be performed using a device of the present invention but are not so limited and may be performed using other devices. Similarly, a system of the present invention may advantageously include a device of the present invention, but is not so limited and may include other devices.

Having described the many embodiments of the present invention in detail, it will be apparent that modifications and variations are possible without departing from the scope of the present invention defined in the appended claims. Furthermore, it should be appreciated that all examples in the present disclosure, while illustrating many embodiments of the present invention, are provided as non-limiting examples and are, therefore, not to be taken as limiting the various aspects so illustrated.

While the present invention has been disclosed with references to certain embodiments, numerous modifications, alterations and changes to the described embodiments are possible without departing from the scope and spirit of the present invention, as defined in the appended claims. Accordingly, it is intended that the present invention not be limited to the described embodiments, but that it have the full scope defined by the language of the following claims, and equivalents thereof. References cited herein are also incorporated by reference as though fully set forth herein. The present invention may be better understood by referring to the following non-limiting examples.

### Example 1

### Open Arthrotomy Testing

In preparation stages for open arthrotomy, EMG needles/sensors (with unsterile lead) are placed on the patient prior to draping and sterile field preparation. The EMG needles/sensors should remain in clear view after draping (transparent dressing). In some cases, testing may be performed in advance of the day of surgery, for example in an office setting facilitated by concomitant use of minimally invasive arthroscopic equipment.

The ACL is identified using the arthroscope or the ACL is carefully exposed using an arthrotomy. The stimulator (e.g., Checkpoint Surgical, Model #9014 Surgical Nerve Stimulator/Locator or B. Brain Medical, Stimuplex HNS12 Peripheral Nerve Stimulator with SENSe) is then anchored in the mid-substance of the ACL.

The leads from the stimulator are connected to the device and sterile EMG leads are connected outside the sterile field to the EMG needles/sensors.

To test the reflex, an electrical stimulus is delivered to the ACL, and EMG feedback is detected in the hamstring muscles (may also be accompanied by visual contraction of muscle). An electrical stimulus is applied that is sufficient to elicit the normal reflex and below the threshold reported to cause pain, as described in previous studies: e.g., three rectangular pulses of 1 ms duration, 8 mA intensity, and 50 Hz frequency; a train of 1-8 monopolar stimuli with an interstimulus interval of 5 ms with a stimulus (amplitude <10V).

The latency of the signal confirms reflex activity (90-10 ms, mean 120 ms). e.g., latency of ˜120 ms, as measured in semitendinosus muscle validating a genuine reaction from artifact (true positive).

If the reflex is not present, the experimental setup is examined for connectivity issues. The procedure is repeated with the knee flexed to 30 degrees as the surgeon performs controlled manipulation of the joint producing antero-posterior translation.

If the reflex is still absent, then the neuromuscular feedback is deemed to be impaired.

### Example 2

### Arthroscopic Testing

In arthroscopic procedures, a positive reflex (i.e., positive EMG feedback with or without visible contraction of the hamstring muscles) in an arthroplasty patient may indicate that the patient is a good candidate to: i) limit the extent of the replacement (compartmentalized arthroplasty in cases of compartmental osteoarthritis); ii) introduce less constraint in the prosthesis, and iii) may have a good prognostic value in terms of comfort and activity level. Positive reflex encourages the use of ACL-sparing treatment algorithms.

Negative reflex (i.e., lack of EMG feedback or EMG feedback that does not fulfill the criteria of being mediated by reflex activity) indicates that the ACL, although intact, does not provide neuromuscular feedback to stabilize the knee. Therefore the operating surgeon should take this into account in choosing an ACL-sparing treatment algorithm if it is perceived that keeping ACL in the treatment strategy is of biomechanical/proprioceptive advantage. For example, in a knee that has no ACL reflex activity, a cruciate-retaining implant is unlikely to provide proprioceptive and biomechanical advantage and a more conventional total knee replacement system may be more appropriate.

### Example 3

### Soft Tissue Reconstruction (Cartilage and Meniscal Restoration)

A positive reflex indicates that the joint has reflex neuromuscular function. Absence of the reflex may have contributed to the existing pathology, e.g., medial meniscal tear. The potential of this device for clinical trials in this area is to guide rehabilitation and identify the patients that can return to sport sooner, as opposed to those who need more neurorehabilitation and programming of protective strategies.

### Example 4

### ACL Impaired

After initial surgical exposure of the anterior cruciate ligament (ACL), the stimulating device is set at 0.5 mA and 0 pulse width. Next, the stimulating tip is placed in direct contact with the ACL and the pulse width is slowly increased until initial muscle contraction of the hamstrings is observed. The physician approximates and records the stimulation parameters required to attain muscle response.

If patients exhibit poor response to stimulation compared to normal threshold to elicit muscle response, sensory function of ACL is impaired and the ACL should be removed and a knee design chosen that accounts for ACL absence (e.g., cruciate-retaining/posterior stabilized instead of bicruciate-retaining knee arthroplasty).

### Example 5

### ACL Randomized Controlled Trials

The device may be used on the ACL in the setting of unicompartmental/robot assisted arthroplasty and soft tissue reconstruction. At the same time, patients undergoing soft tissue reconstruction in their knee (including ACL reconstruction) are able to undergo testing including ACL remnant following ACL rupture where a positive reflex may advocate for specialized remnant incorporation ACL reconstruction, with the goal of restoring the protective ACL-hamstring reflex.

A high throughput surgical center tests the device and concept in patients with ligament-sparing arthroplasty techniques to correlate pre-surgical neuromuscular status with advanced surgical outcomes.

We claim:

1. A method for testing neuromuscular status of a ligamentous or soft tissue structure in a mammal, comprising:
(a) stimulating a ligamentous or soft tissue structure;
(b) observing EMG output to determine a quantitative measure of neuromuscular status for the ligamentous or soft tissue structure; and
(c) using the quantitative measure of neuromuscular status to guide medical treatment related to the ligamentous or soft tissue structure.

2. The method of claim 1, wherein the stimulating is via extrinsic electrical stimulus.

3. The method of claim 1, wherein the quantitative measure of neuromuscular status is binary.

4. The method of claim 1, wherein the stimulating is via physical manipulation of a joint structure.

5. The method of claim 1, wherein the ligamentous structure is the anterior cruciate ligament.

6. The method of claim 1, wherein the ligamentous structure is the posterior cruciate ligament.

7. The method of claim 1, wherein stimulating comprises anteroposterior translation of the knee.

8. The method of claim 1, wherein the ligamentous structure is the coracoacromial ligament.

9. The method of claim 1, wherein the stimulating is performed before, during or after a surgical procedure.

10. A device for testing neuromuscular status of a mammalian joint comprising a component for stimulating a ligamentous or soft tissue structure, a component for detecting neuromuscular reflex activity to generate electromyographic (EMG) output, and a processing unit component to receive and process the EMG data.

11. The device of claim 10, wherein stimulation is via electrical stimulus.

12. The device of claim 10, further comprising a stimulator extension for electrical stimulation in arthroscopic applications.

13. The device of claim 10, further comprising a display for a quantitative measure of the neuromuscular status of the ligamentous or soft tissue structure

14. The device of claim 10, further comprising a component for detecting reflex latency.

15. The device of claim 10, wherein the stimulating component comprises a member for contacting a ligamentous structure without damaging the ligamentous structure.

16. The device of claim 10, wherein the feedback component comprises an electrode.

17. A system for testing neuromuscular status of a ligamentous or soft tissue structure in a mammal, comprising a stimulating component, a processing component, and a feedback component to indicate a quantitative measure of neuromuscular status for the ligamentous or soft tissue structure.

18. The system of claim 17, wherein the stimulating component comprises a device for electrical stimulation.

19. The system of claim 17, further comprising an extension for electrical stimulation in arthroscopic applications.

20. The system of claim 17, wherein the feedback component detects neuromuscular reflex activity to generate electromyographic (EMG) output.

21. The system of claim 17, wherein the feedback component comprises an electrode.

22. The system of claim 17, wherein the feedback component detects reflex latency.

23. The system of claim 17, further comprising computer readable media for controlling strength, duration, and/or frequency of electrical stimulation.

24. The system of claim 17, further comprising a processing unit for controlling stimulation, detecting and processing feedback, and generating a quantitative measure of neuromuscular status related to electromyographic (EMG) output and/or absolute reflex latency based on detected neuromuscular reflex activity.

25. The system of claim 24, wherein the stimulating component and feedback component are in communication with the processing unit.

26. The system of claim 17, further comprising computer readable media for controlling stimulation, detecting and processing feedback, and generating a quantitative measure of neuromuscular status related to electromyographic (EMG) output and/or absolute reflex latency based on detected neuromuscular reflex activity.

27. The system of claim 26, wherein the stimulating component and feedback component are in communication with the computer readable media.

28. The system of claim 17, wherein the feedback is a quantitative or binary measure representative of neuromuscular status.

29. A method for individualizing a treatment plan comprising: neuromuscular testing according to the method of claim 1, further comprising refining a treatment plan before, during, or after a surgical procedure as a result of determined neuromuscular status.

30. Computer readable media for testing neuromuscular status using the method of claim 1.
